(19) United States
(12) Patent Application Publication
(10) Pub. No.: US 2026/0199582 A1
(43) Pub. Date: Jul. 16, 2026

(54) EXPANDABLE ASPIRATION SHEATH WITH EXPANSION SLEEVE

(71) Applicant: Boston Scientific Scimed, Inc., Maple Grove, MN (US)

(72) Inventors: Joseph Stanley Czyscon, Elk River, MN (US); Derek Kenneth Larson, Golden Valley, MN (US)

(73) Assignee: Boston Scientific Scimed, Inc., Maple Grove, MN (US)

(21) Appl. No.: 19/447,325

(22) Filed: Jan. 13, 2026

Related U.S. Application Data
(60) Provisional application No. 63/744,940, filed on Jan. 14, 2025.

(51) Int. Cl. A61M 1/00; A61M 25/00; A61M 29/00; A61B 17/22; A61M 25/06

(52) U.S. Cl. CPC A61M 1/87; A61M 25/0045; A61M 25/0054; A61M 25/0074; A61M 29/00; A61B 2017/22079; A61M 2025/0681; A61M 2205/0216

(57) ABSTRACT

An aspiration sheath system is disclosed. The aspiration sheath system includes an expandable sheath having a proximal end region, a distal end region and a lumen extending therebetween. The distal end region has a first diameter in a delivery configuration smaller than a first diameter of the proximal end region in the delivery configuration. The system further includes a dilator and an expansion sleeve insertable within the lumen of the expandable sheath. The dilator is insertable thought he lumen of the expandable sheath without expanding the proximal end region and the distal end region. The expansion sleeve insertable within the lumen of the expandable sheath upon removal of the dilator therefrom to expand the distal end region from the first diameter of the distal end region to a second diameter of the distal end region larger than the first diameter of the distal end region.

### CROSS-REFERENCE TO RELATED APPLICATION

This application claims the benefit of U.S. Provisional Patent Application Serial No. 63/744,940, filed January 14, 2025, entitled " EXPANDABLE ASPIRATION SHEATH WITH EXPANSION SLEEVE”, which is incorporated by reference herein in its entirety.

### TECHNICAL FIELD

The present disclosure relates generally to medical devices and more particularly to expandable aspiration sheaths that are adapted for aspirating emboli and/or thrombus.

### BACKGROUND

In some instances, it may be desirable to use a medical device for aspirating emboli and/or thrombus from a vasculature. Inserting the medical devices into the vasculature and other navigable anatomical structures may result in undesirable forces being applied to vessel and/or structural walls. For example, as the medical device passes into the vasculature, it may make undesirable contact with one or more vessel walls. This interference may cause injury to the vessel. Vessel trauma resulting from forces applied to the vessel wall by the aspiration may be lessened by minimizing the size of the medical device used to access the vessel. However, reducing the size of the medical device also reduces the size of the aspiration lumen through the medical device, limiting aspiration through the medical device. Therefore, it may be desirable to design an aspiration sheath having a reduced insertion profile, yet capable of expansion within the vasculature to provide a larger lumen for aspiration therethrough.

### BRIEF SUMMARY

This disclosure provides design, material, systems, manufacturing method, and use alternatives for dilators, sheaths, sleeves and other related devices and device systems. An example medical device system of the present disclosure is an aspiration sheath system. The system includes an expandable sheath having a proximal end region, a distal end region and a lumen extending therebetween. The distal end region has a first diameter in a delivery configuration smaller than a first diameter of the proximal end region in the delivery configuration. The system also includes a dilator insertable within the lumen of the expandable sheath and through the proximal end region and the distal end region, without expanding the proximal end region and the distal end region. The system also includes an expansion sleeve insertable within the lumen of the expandable sheath upon removal of the dilator therefrom. The expansion sleeve is configured to expand the distal end region from the first diameter of the distal end region to a second diameter of the distal end region larger than the first diameter of the distal end region.

Alternatively or additionally to any of the examples disclosed herein, the first diameter of the distal end region is 18F or less and the second diameter of the distal end region is 20F or more.

Alternatively or additionally to any of the examples disclosed herein, the distal end region is retractable toward the first diameter of the distal end region after withdrawing the expansion sleeve.

Alternatively or additionally to any of the examples disclosed herein, the expansion sleeve maintains expansion of the distal end region in the second diameter during aspiration through the expandable sheath.

Alternatively or additionally to any of the examples disclosed herein, the expansion sleeve includes an aspiration lumen extending therethrough.

Alternatively or additionally to any of the examples disclosed herein, the distal end region includes: an inner layer defining the lumen of the expandable sheath, an intermediate layer surrounding the inner layer, and an outer layer surrounding the intermediate layer.

Alternatively or additionally to any of the examples disclosed herein, the intermediate layer comprises a cut pattern.

Alternatively or additionally to any of the examples disclosed herein, the cut pattern comprises a plurality of discontinuous slits alternating with frangible links.

Alternatively or additionally to any of the examples disclosed herein, the frangible links are configured to break upon initial expansion of the distal end region to the second diameter.

Alternatively or additionally to any of the examples disclosed herein, the cut pattern extends in a helical direction along the distal end region.

Alternatively or additionally to any of the examples disclosed herein, the outer layer is formed of an elastomeric material configured to apply a radially inward force on the intermediate layer when the expansion sleeve is removed to return the distal end region toward the delivery configuration.

Alternatively or additionally to any of the examples disclosed herein, in the delivery configuration, the lumen of the expandable sheath has a first diameter throughout the proximal end region and a second diameter throughout the distal end region, the second diameter of the lumen being less than the first diameter of the lumen in the delivery configuration.

Alternatively or additionally to any of the examples disclosed herein, the dilator has a first diameter along a proximal end region of the dilator and the dilator has a second diameter along a distal end region of the dilator, the second diameter of the dilator being less than the first diameter of the dilator.

Alternatively or additionally to any of the examples disclosed herein, the first diameter of the dilator is greater than the second diameter of the lumen of the expandable sheath in the delivery configuration.

Another example is an aspiration sheath system. The system includes an expandable sheath having a proximal end region, a distal end region and a lumen extending therebetween. The distal end region is radially expandable from a first diameter in a delivery configuration to a second diameter in an expanded configuration. The system also includes a dilator insertable within the lumen of the expandable sheath in the delivery configuration with a distal tip of the dilator extending distally beyond the expandable sheath. The system also includes an expansion sleeve insertable within the lumen of the expandable sheath upon removal of the dilator therefrom. The expansion sleeve is configured to expand the distal end region of the expandable sheath from the first diameter to the second diameter.

Alternatively or additionally to any of the examples disclosed herein, the distal end region includes: an inner layer defining the lumen of the expandable sheath, an intermediate layer surrounding the inner layer, and an outer layer surrounding the intermediate layer.

Alternatively or additionally to any of the examples disclosed herein, the intermediate layer comprises a cut pattern.

Alternatively or additionally to any of the examples disclosed herein, the cut pattern comprises a plurality of discontinuous slits alternating with frangible links.

Alternatively or additionally to any of the examples disclosed herein, the frangible links are configured to break upon initial expansion of the distal end region to the second diameter.

Another example is a method of using a medical device system. The method includes advancing an expandable sheath through a blood vessel in a delivery configuration with a dilator disposed in a lumen of the expandable sheath and extending distally therefrom. The expandable sheath includes a proximal end region and a distal end region, wherein in the delivery configuration the distal end region has a first outer diameter less than an outer diameter of the proximal end region. The method further includes removing the dilator from the lumen of the expandable sheath, and thereafter, advancing an expansion sleeve through the lumen of the expandable sheath to expand the distal end region from the first outer diameter to a second outer diameter greater than the first outer diameter.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense.

### BRIEF DESCRIPTION OF THE SEVERAL VIEWS OF THE DRAWINGS

The drawings illustrate the design and utility of preferred embodiments of the present disclosure. It should be noted that the figures are not drawn to scale and that elements of similar structures or functions are represented by like reference numerals throughout the figures. In order to better appreciate how the above-recited and other advantages and objects of the present disclosure are obtained, a more particular description of the present disclosure briefly described above will be rendered by reference to specific embodiments thereof, which are illustrated in the accompanying drawings. Understanding that these drawings depict only exemplary embodiments of the disclosure and are not therefore to be considered limiting of its scope, the present disclosure will be described and explained with additional specificity and detail through the use of the accompanying drawings, in which:

FIG. 1 illustrates a side view of an aspiration sheath system, including an expandable aspiration sheath in a delivery configuration, and associated components.

FIG. 2A illustrates a side view of a dilator of the aspiration sheath system of FIG. 1.

FIG. 2B illustrates a side view of an expandable sleeve of the aspiration sheath system of FIG. 1.

FIG. 3 illustrates a side view of the aspiration sheath system of FIG. 1, in an expanded configuration.

FIG. 4 is a cross-sectional view of the aspiration sheath system of FIG. 3 taken along line 4-4.

FIG. 5 is a perspective view of the distal expandable region of the aspiration sheath having portions cut away for illustrative purposes.

FIG. 6 is a perspective view of the distal expandable region of the aspiration sheath having the outer layer removed for illustrative purposes.

FIG. 7 is a side view of the distal expandable region of the aspiration sheath being radially expanded.

FIGS. 8 and 9 illustrate an exemplary method of using the aspiration sheath system of FIG. 1.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5). As used in this disclosure and appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following detailed description should be read with reference to the drawings in which similar elements in different drawings are numbered the same. The drawings, which are not necessarily to scale, depict illustrative examples and are not intended to limit the scope of the disclosure.

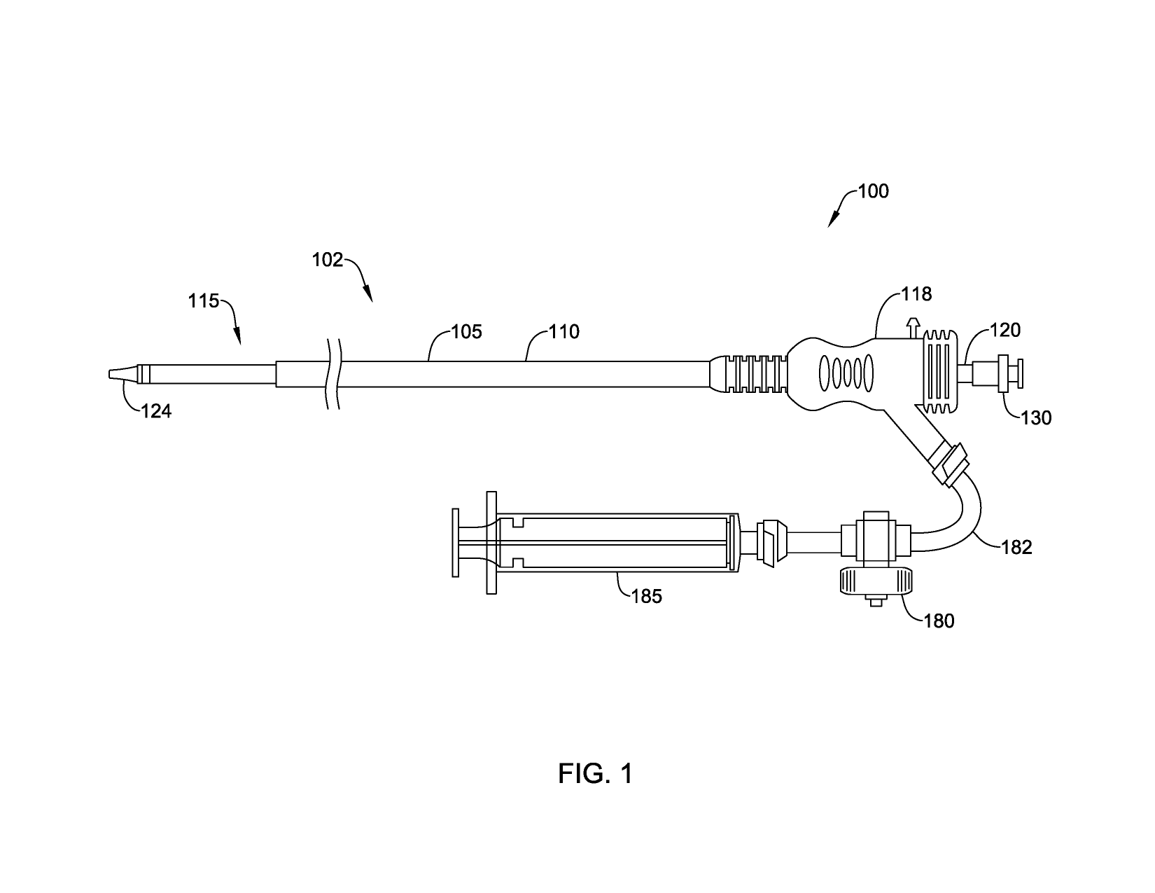

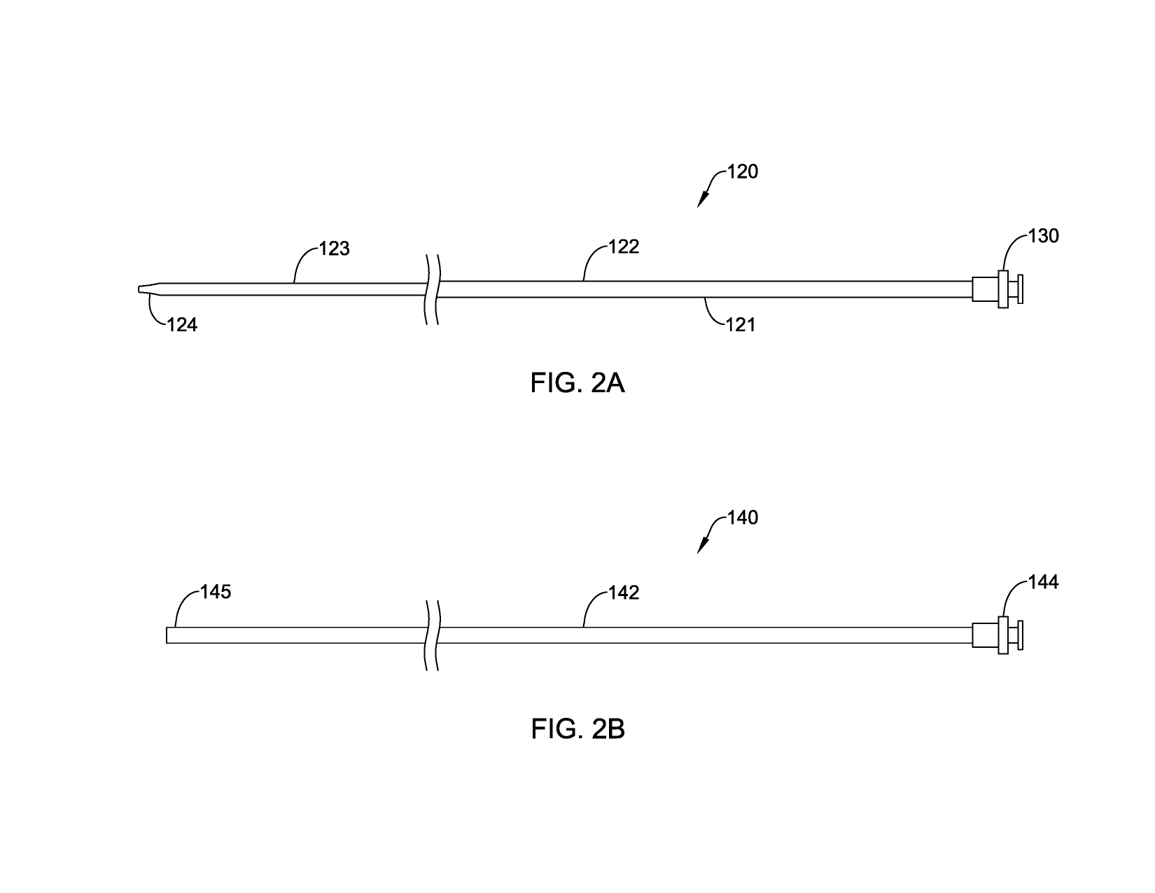

As discussed above, it may be desirable to design an aspiration sheath having a reduced insertion profile, yet capable of expansion within the vasculature to provide a larger lumen for aspiration therethrough. FIG. 1 is a side view of components of an example aspiration sheath system 100 of the present disclosure. FIG. 2A illustrates a side view of a dilator of the aspiration sheath system 100 of FIG. 1 and FIG. 2B is a side view of an expansion sleeve 140 of the aspiration sheath system 100. As shown in FIG. 1, the aspiration sheath system 100 may include an aspiration sheath 102 having an elongate shaft 105 extending distally from a manifold 118. The elongate shaft 105 may have a proximal end region 110 and a distal end region 115 extending distally of the proximal end region 110. The proximal end region 110 may extend to and be coupled or otherwise connected to the manifold 118. The distal end region 115 may extend to a distal end of the elongate shaft 105. A lumen (not shown in FIG. 1) may extend through the elongate shaft 105 from the proximal end region 110 to the distal end region 115, and thus the lumen may extend through the proximal end region 110 and the distal end region 115 of the elongate shaft 105. The elongate shaft 105 may include a single lumen or a plurality of lumens disposed concentrically, coaxially, in a side-by-side relationship, in an array, or in any configuration feasible for the passage of medical devices and/or like devices. The distal end region 115 of this and any other examples may further include a distal tip defining a distal opening into the lumen of the elongate shaft 105. In some instances, the distal tip may include radiopaque material. For instance, and applicable to all examples of the present disclosure, radiopaque material may be applied to the distal tip or any other part of the elements disclosed herein through any desired process.

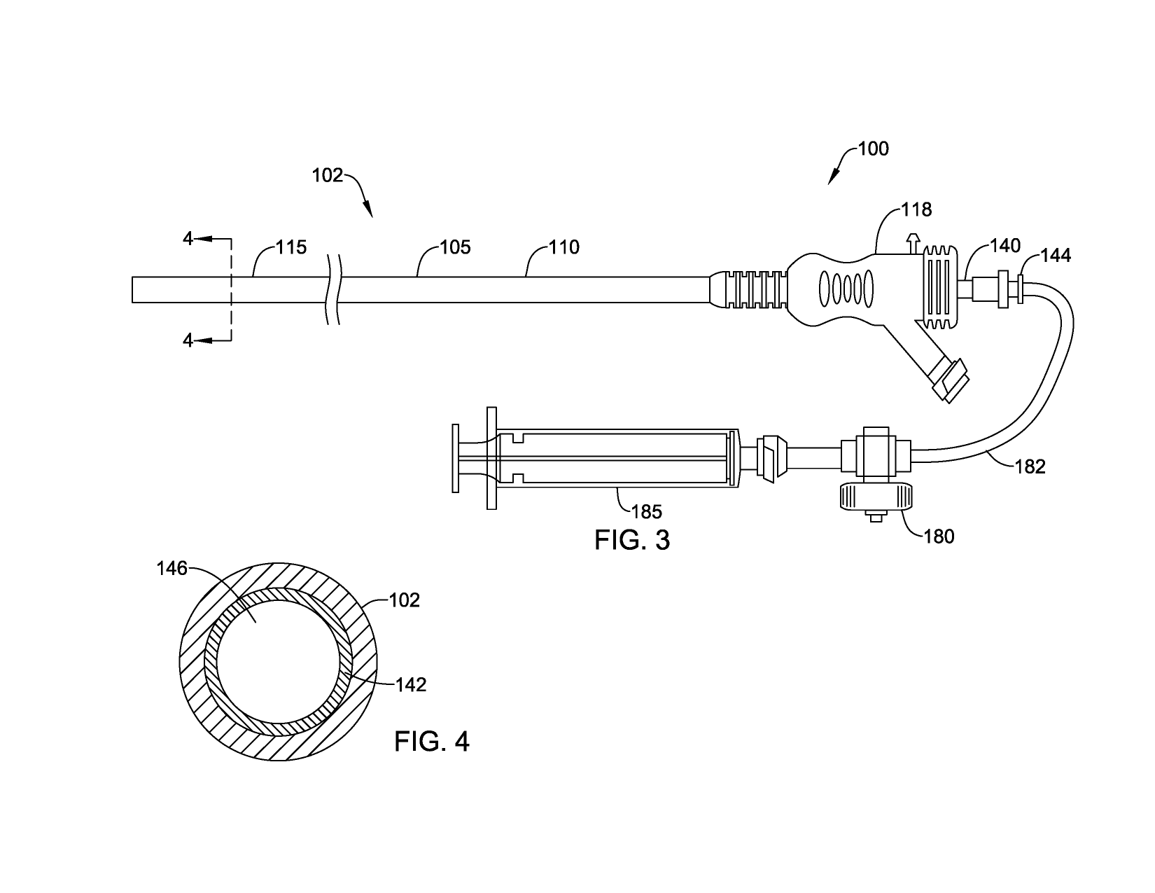

As shown in FIG. 1, distal end region 115 of the elongate shaft 105 of the aspiration sheath 102 may be a radially expandable region that is expandable from a reduced diameter delivery configuration, shown in FIG. 1, to a radially expanded configuration, shown in FIG. 3. The radially expandable region of the distal end region 115 may extend along the distal end region 115 to the distal end of the elongate shaft 105. In some instances, the proximal end region 110 extending proximal of the distal end region 115 may be a non-expandable section of the elongate shaft 105. In other words, the proximal end region 110 extending proximal of the expandable distal end region 115 may not be readily radially expandable, unlike the distal end region 115. As shown in FIG. 1, the distal end region 115 is in a radially collapsed, or reduced diameter configuration. In the overall configuration of FIG. 1 (i.e., when the distal end region 115 is in a radially contracted, or reduced diameter delivery configuration), the proximal end region 110 of the elongate shaft 105 may have a relatively large outer diameter or French (Fr) size upon access, such as 20 Fr. or greater, 22 Fr. or greater, or 24 Fr. or greater. However, other French sizes are contemplated for the outer diameter of the proximal end region 110 in this configuration. Further shown in the overall configuration of FIG. 1, the expandable distal end region 115, while in the radially collapsed, reduced diameter configuration, may have an outer diameter or French size less than that of the outer diameter or French size of the proximal end region 110. For instance, in some instances the distal end region 115 may have a French size of 16 Fr. or less, 14 Fr. or less, 12 Fr. or less, or 10 Fr. or less, for example. However, other French sizes are contemplated for the distal end region 115 while in the radially collapsed, reduced diameter configuration.

The aspiration sheath system 100 may also include a dilator 120, also shown in FIG. 2A, configured to be inserted through the lumen of the elongate shaft 105 of the aspiration sheath 102 in the radially collapsed, reduced diameter configuration. The dilator 120 may facilitate introduction of the aspiration sheath 102 into the vasculature and subsequent advancement of the aspiration sheath 102 through the vasculature of the patient. The dilator 120 may include an elongate shaft 122 extending distally from a hub 130 to a distal tip 124 of the dilator 120. The elongate shaft 122 of the dilator 120 may extend distally beyond the distal end of the elongate shaft 105 while the hub 130 is positioned proximal of the manifold 118. For example, the dilator 120 may include a tapered distal tip 124 extendable distally beyond the distal tip of the elongate shaft 105 of the aspiration sheath 102 to facilitate delivery of the aspiration sheath 102 through the vasculature of a patient. The dilator 120 may include a proximal end region 121 having a first outer diameter and a distal end region 123 having a second outer diameter less than the first outer diameter of the proximal end region 121. The outer diameters of the dilator 120 may correspond to the different inner diameters (i.e., the different diameters of the lumen through the elongate shaft 105) of the proximal end region 110 and the distal end region 115 of the elongate shaft 105 of the aspiration sheath 102 in its radially collapsed, reduced diameter delivery configuration. In other words, the proximal end region 121 of the elongate shaft 122 of the dilator 120 may be positionable within the proximal end region 110 of the elongate shaft 105 having a first inner diameter and the distal end region 123 of the elongate shaft 122 of the dilator 120 may be positionable within the distal end region 115 of the elongate shaft 105 having a second inner diameter less than the first inner diameter in the radially collapsed, reduced diameter delivery configuration. The diameter of the proximal end region 121 of the dilator 120 may be greater than the second inner diameter of the distal end region 115 of the elongate shaft 105 in the radially collapsed, reduced diameter delivery configuration.

The aspiration sheath system 100 may also include a syringe 185, connected to a port of the manifold 118 via a length of tubing 182. A valve 180, such as a stop cock valve, may be positioned between the syringe 185 and the tubing 182 that may be selectively opened and closed to fluidly couple the syringe 185 to the lumen of the tubing 182, and thus fluidly couple the syringe 185 to the lumen of the elongate shaft 105 of the aspiration sheath 102. The syringe 185 may be used to create a vacuum for aspirating emboli and/or thrombi through the aspiration sheath 102 and into the syringe 185.

FIG. 2B is a side view of an expansion sleeve 140 of the aspiration sheath system 100 configured to be inserted through the lumen of the elongate shaft 105 of the aspiration sheath 102 upon removal of the dilator 120. The expansion sleeve 140 may include an elongate shaft 142 extending distally from a hub 144 to a distal end 145 of the expansion sleeve 140. After inserting the aspiration sheath 102 into a vasculature to a desired treatment location, the dilator 120 of FIG. 2A may be removed from the lumen 125 of the elongate shaft 105 of the aspiration sheath 102, and thereafter, the expansion sleeve 140 may be advanced distally through the lumen of the elongate shaft 105 of the aspiration sheath 102. FIG. 3 illustrates the expansion sleeve 140 inserted through the elongate shaft 105 of the aspiration sheath 102 after removal of the dilator 120 therefrom. In some instances, the elongate shaft 142 of the expansion sleeve 140 may extend to the distal end of the elongate shaft 105 or distally beyond the distal end of the elongate shaft 105 while the hub 144 is positioned proximal of the manifold 118.

Insertion of the elongate shaft 142 of the expansion sleeve 140 through the elongate shaft 105 of the aspiration sheath 102 causes the distal end region 115 of the elongate shaft 105 to radially expand to an expanded configuration, shown in FIG. 3. In the overall configuration of FIG. 3 (i.e., when the radially expandable distal end region 115 is in a radially expanded configuration), the distal end region 115 of the elongate shaft 105 may have an expanded outer diameter or French size greater than its outer diameter or French size in the radially collapsed, reduced diameter configuration. For example, the distal end region 115 may have a French size such as 18 Fr. or more, 20 Fr. or more, 22 Fr. or more, or 24 Fr. or more in the radially expanded configuration. However, other French sizes are contemplated for the distal end region 115 in this radially expanded configuration. In some instances, as shown in FIG. 3, the distal end region 115, while in the radially expanded configuration, may have an outer diameter or French size equal to that of the outer diameter or French size of the proximal end region 110. In other instances, the distal end region 115 may have an outer diameter less than or greater than the outer diameter of the proximal end region 110 in the radially expanded configuration.

In some instances, the distal end region 115 may return to or toward its radially collapsed, reduced diameter configuration upon removal of all external forces subsequent to being radially expanded to the radially expanded configuration. For example, upon removal of the elongate shaft 142 of the expansion sleeve 140 therefrom, the distal end region 115 of the elongate shaft 105 of the aspiration sheath 102 may revert to a radially retracted configuration having an outer diameter or French size less than that in its radially expanded configuration shown in FIG. 3, but greater than that in its radially collapsed configuration shown in FIG. 1. In other words, the outer diameter of the distal end region 115 in its radially retracted configuration may be between the outer diameter of the distal end region 115 in its radially collapsed, delivery configuration and the outer diameter of the distal end region 115 in its radially expanded configuration.

FIG. 4 is a cross-sectional view of the elongate shaft 105 of the aspiration sheath 102 with the elongate shaft 142 of the expansion sleeve 140 inserted therein. The outer surface of the elongate shaft 142 may be in direct contact with the inner surface of the elongate shaft 105 of the aspiration sheath 102, while the elongate shaft 142 of the expansion sleeve 140 defines an aspiration lumen 146 extending therethrough. The syringe 185, or another syringe or other vacuum device, may be connected to the hub 144 of the expansion sleeve 140 via a length of tubing 182. A valve 180, such as a stop cock valve, may be positioned between the syringe 185 and the tubing 182 that may be selectively opened and closed to fluidly couple the syringe 185 to the lumen of the tubing 182, and thus fluidly couple the syringe 185 to the aspiration lumen 146 of the elongate shaft 142 of the expansion sleeve 140. The syringe 185 may be used to create a vacuum for aspirating emboli and/or thrombi through the aspiration lumen 146, and thus through the elongate shaft 105 of the aspiration sheath 102, and into the syringe 185.

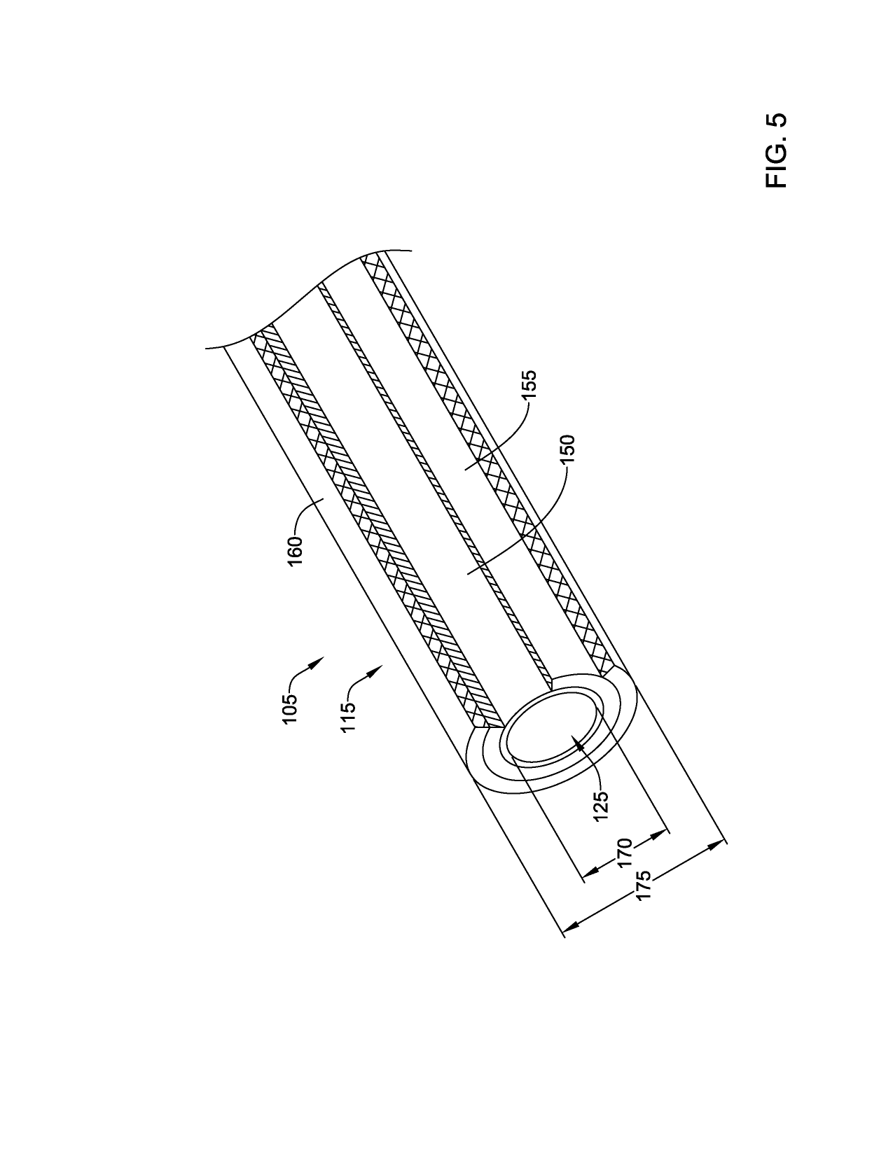

FIG. 5 is an enlarged partially cut-away perspective view of the expandable distal end region 115 of the elongate shaft 105 of the aspiration sheath 102 that is applicable to any and all examples of the present disclosure. FIG. 5 shows a close-up and cutaway view of the elongate shaft 105 and its layered construction. Elongate shaft 105 overall possesses an inner diameter 170 defining a lumen 125 of the elongate shaft 105 and an outer diameter 175. Elongate shaft 105 of this and other examples may include one or more layers, or a plurality of layers, and more particularly may include two or more layers, three or more layers, or four or more layers. In this and other examples, the elongate shaft 105 may include three layers: inner layer 150 (which may define the inner surface of the elongate shaft 105), intermediate layer 155, and outer layer 160 (which may define the outer surface of the elongate shaft 105). The intermediate layer 155 may be disposed between the inner layer 150 and the outer layer 160. As shown in FIG. 5, all layers of the elongate shaft 105 may concentrically surround the lumen 125 and longitudinal axis of the elongate shaft 105. Any of the layers described herein may be formed of the same material, different materials, a mixture of materials, a blend of materials, a patterned arrangement of materials (i.e. two layers of the same material, one layer of different material), a braid of materials, or the like. The layers may be bonded, affixed, adhered, or otherwise coupled to each other by any means or methods known in the art. Other material types, forms, and configurations are further contemplated and will be discussed herein.

The outer layer 160 of this and other examples may include an elastomeric material that is elastically deformable when the expandable distal end region 115 of the elongate shaft 105 radially expands to its radially expanded configuration. In some instances, the elasticity of the outer layer 160 may provide a radially inward force on the intermediate layer 155 and the inner layer 150 by virtue of the material properties of the elastomeric material, causing the distal end region 115 to at least partially radially contract when not subjected to a radially outward force exerted by the expansion sleeve 140 when disposed therein. In other words, the outer layer 160 of this and other examples may be formed of one or more elastomeric materials or like materials which are elastically deformable (i.e., able to reverse and/or revert form and/or shape under applied stress) such that the outer layer 160 may apply a radially inward force on the intermediate layer 155, particularly in the instance and particular to the portions of the distal end region 115 that are no longer in intimate contact with the expansion sleeve 140 disposed therein, an expansion dilator disposed therein, or other like device. For example, the outer layer 160 may be formed of a thermoplastic polyurethane elastomer such as Pellethane®, in some instances.

The inner layer 150 may be a highly lubricious layer in some instances to facilitate advancing a medical device therethrough. For example, the inner layer 150 may be formed of polytetrafluoroethylene (PTFE), such as etched ePTFE, in some instances.

The intermediate layer 155 may be a polymer material, such as polyamide, a polyether block polyamide (PEBA), or the like.

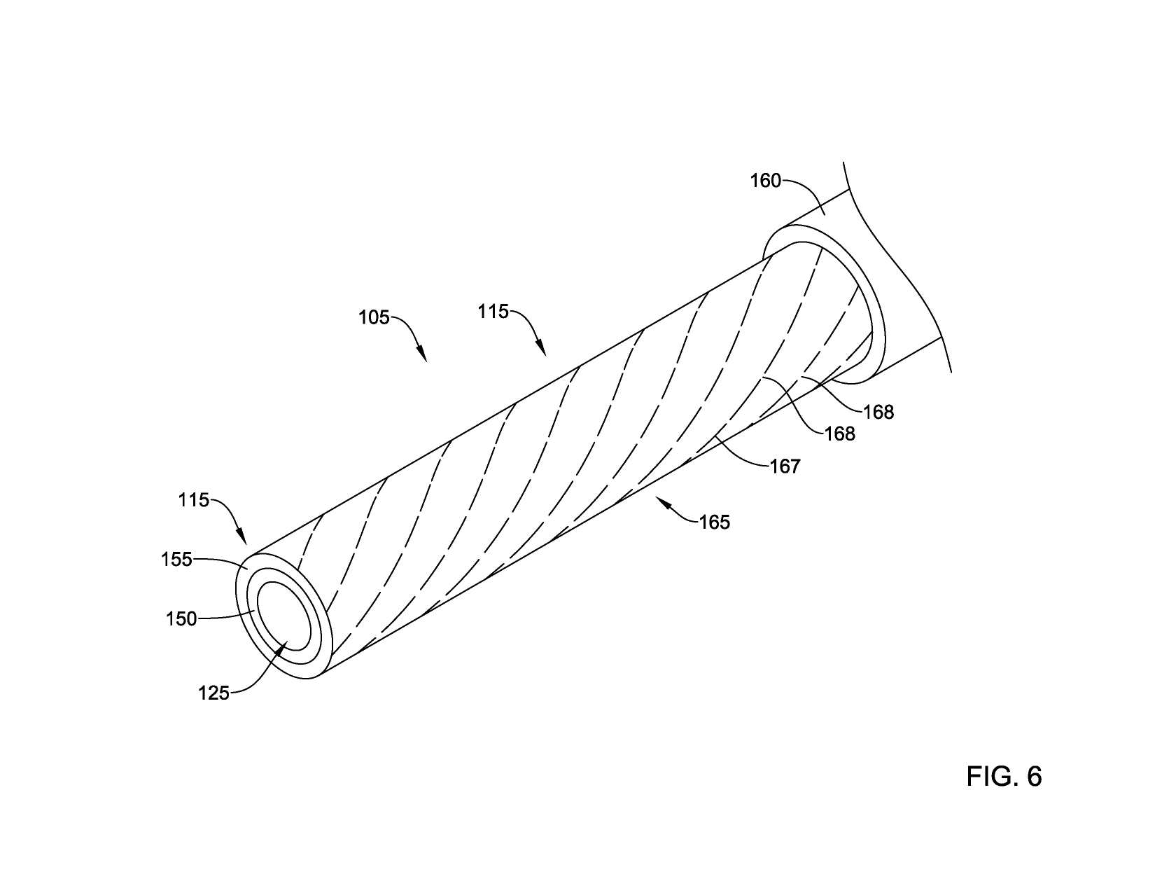

FIG. 6 is an enlarged perspective view of the distal end region 115 of the elongate shaft 105 of the aspiration sheath 102 with the outer layer 160 removed, to illustrate a cut pattern 165 formed in the intermediate layer 155 and/or inner layer 150 of the elongate shaft 105. As shown in FIG. 6, cut pattern 165 may extend along the expandable distal end region 115 of the elongate shaft 105 in a helical direction. However, other directions and cut patterns are contemplated. Other cut directions contemplated include, but are not limited to: serpentine cut directions, sinusoidal cut directions, hyperbolic cut directions, linear cut directions, curvilinear cut directions, lateral cut directions, radial cut directions, proximal cut directions, distal cut directions, or any combination or permutation of the aforementioned or any of the like.

The cut pattern 165 may be formed in the intermediate layer 155 and/or inner layer 150 prior to applying the outer layer 160. For example, the outer layer 160 may be extruded over, reflowed, or otherwise positioned over the intermediate layer 155 after forming the cut pattern 165.

Alternatively or additionally, various cut patterns are also contemplated. Cut patterns may include but are not limited to: serpentine cut patterns, sinusoidal cut patterns, hyperbolic cut patterns, jigsaw cut patterns, linear cut patterns, curvilinear cut patterns, prismatic cut patterns, geometric cut patterns, repeating cut patterns, intermittent cut patterns, or any combination or permutation of the aforementioned and/or the like.

Alternatively or additionally, any of the cut patterns described by the examples disclosed herein may be pre-formed cut patterns. In other words, any of the cut patterns described by the examples disclosed herein may be pre-set or pre-formed into one or more of the outer layer 160, intermediate layer 155, and/or inner layer 150. The cut patterns and/or pre-formed cut patterns of the examples disclosed herein may include a plurality of discontinuous slits or slots alternating with a plurality of frangible links which will be further described herein. The plurality of discontinuous slits or slots may alternatively or additionally take various forms including but not limited to: angled slits or slots, perforated slits or slots, curved slits or slots, helical slits or slots, curvilinear slits or slots, linear slits or slots, or any combination or permutation of the aforementioned and/or the like. Alternatively or additionally to any of the examples disclosed herein, the discontinuous slits or slots may take the non-limiting forms of perforations, depressions, grooves, etches, valleys, furrows, concavities, knurls or any combination or permutation of the aforementioned and/or the like.

As shown in FIG. 6, the cut pattern 165 may include a plurality of slits or slots 167 alternating with frangible link 168 along a helical direction. In other words, the cut pattern 165 may include a frangible link 168 between adjacent slits or slots 167, defining discontinuous helical cuts along the cut pattern 165 of the expandable distal end region 115 of the elongate shaft 105. The frangible links 168 may be configured to break or separate as the distal end region 115 is radially expanded from the radially collapsed, reduced diameter delivery configuration to the radial expanded configuration for the first time. In other instances, the links between adjacent slits or slots may be made of an elastomeric material adapted to stretch and aid in the expansion of the distal end region 115 into its radially expanded configuration while also allowing the distal end region 115 to retract toward or to its radially retracted, reduced diameter configuration by virtue of the elastomeric properties of the elastomeric or similar material incorporated as will be described further herein. It is noted that the frangible links 168 may be provided between adjacent slits or slots of the cut pattern, regardless of the configuration of the cut pattern to permit radial expansion of the distal end region 115.

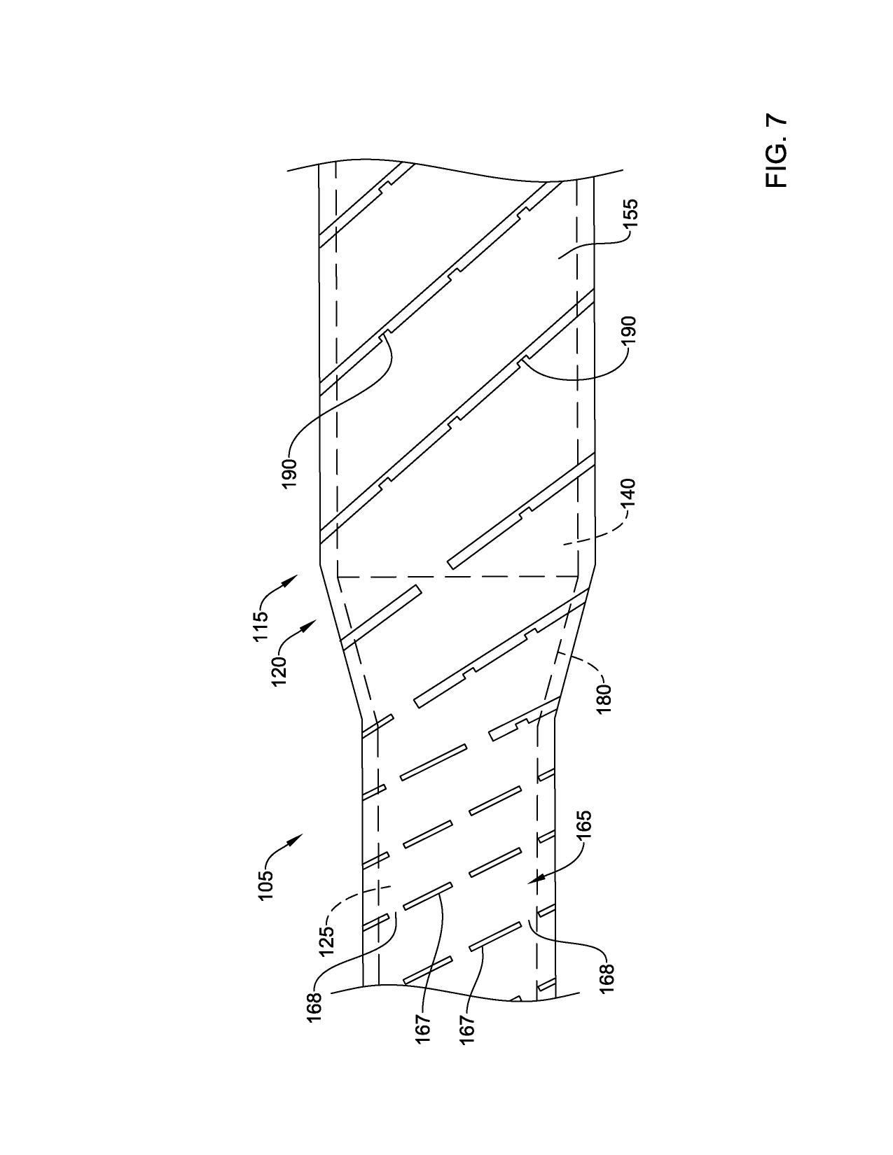

FIG. 7 is an enlarged perspective view of the distal end region 115 of the elongate shaft 105 of the aspiration sheath 102 as the expansion sleeve 140, or another device, is guided through the lumen 125 of the distal end region 115. The expansion sleeve 140 may be used to engage the expandable distal end region 115 from the interior of the distal end region and as the expansion sleeve 140 is pushed through the distal end region 115, the expansion sleeve 140 contacts the interior wall of the distal end region 115, expanding the cuts of the cut pattern 165 such that they break away, and such that the frangible links 168 break apart, becoming fractured links 190. The fractured links 190, given their material construction, expand due to the internal and radial outward force exerted by the action of the expansion sleeve 140, such that the fractured links 190 may expand farther apart relative to one another and permit radial expansion of the lumen 125 through the distal end region 115 of the elongate shaft 105. The radial outward force exerted by the expansion sleeve 140 also radially expands and elastically stretches the outer layer 160.

As described above, after the expandable distal end region 115 is initially radially expanded to the radially expanded configuration, the radially stretched outer layer 160 may apply a radially inward force on the intermediate layer 155 and the inner layer 150 to return the distal end region 115 to or toward the radially retracted, reduced diameter configuration once the expansion force of the expansion sleeve 140 has been removed by virtue of the material properties of the elastomeric material.

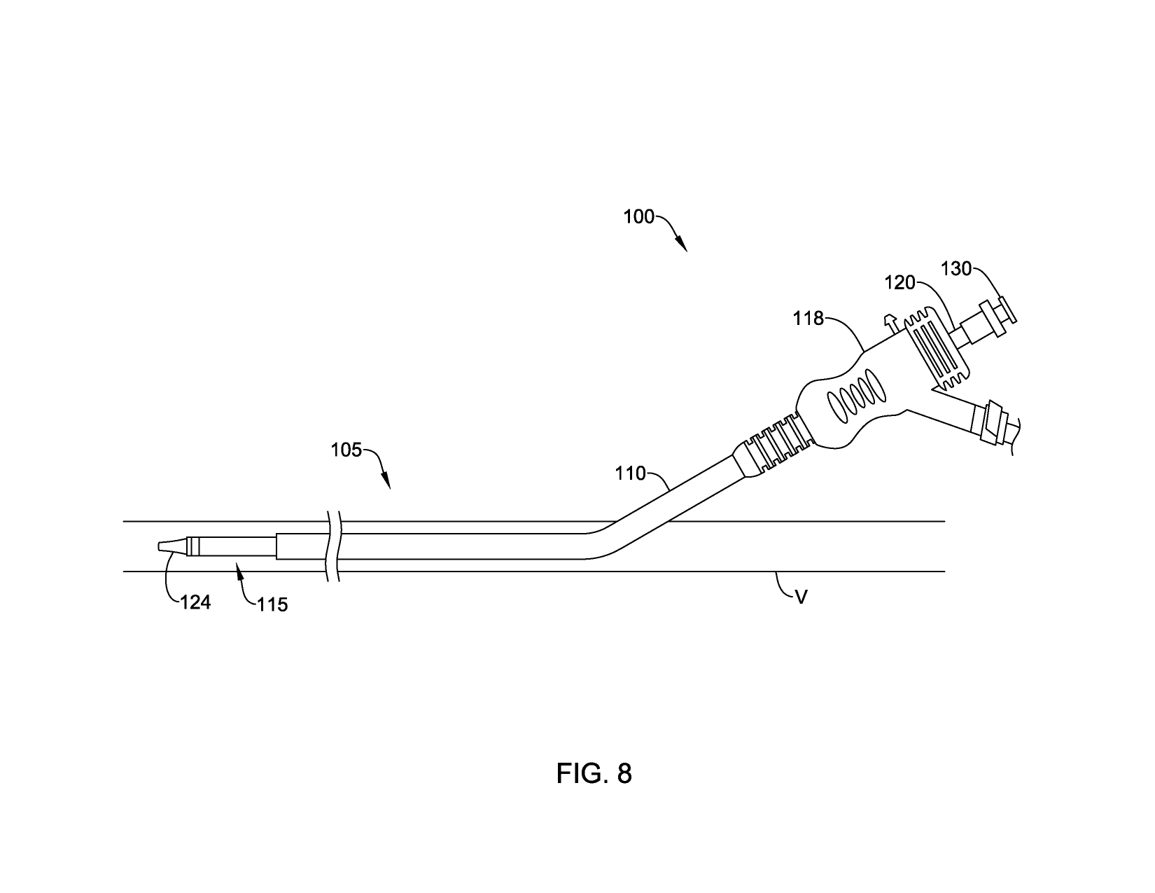

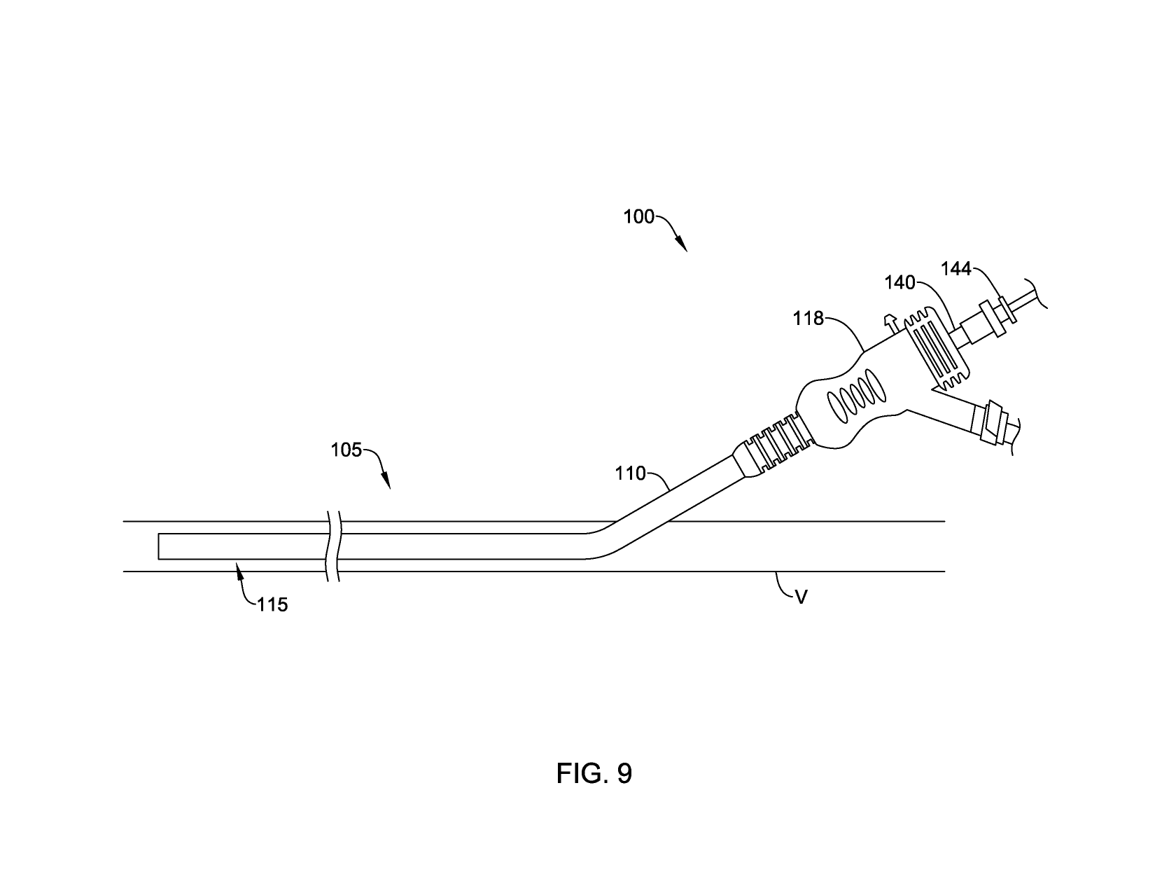

FIGS. 8 and 9 illustrate a sequence of steps of utilizing the aspiration sheath system 100. FIG. 8 is a side view of the aspiration sheath system 100 of the present disclosure as the elongate shaft 105 of the aspiration sheath 102 is positioned within the blood vessel V of a patient or subject with the dilator 120 disposed therein and a distal tip 124 of the dilator 120 extending distally beyond the distal end of the elongate shaft 105. The aspiration sheath system 100 such may be deployed within the blood vessel V (e.g., by tracking along a guidewire already positioned within the blood vessel V), and may be advanced into the blood vessel V with the distal end region 115 of the elongate shaft 105 in a radially contracted configuration. The distal end region 115 of the elongate shaft 105 is provided in a radially contracted configuration such that it provides easy access and initial navigation into the blood vessel V and greater vasculature. Once advanced to a desired location within the blood vessel V, the dilator 120 may be retracted and removed from the elongate shaft 105 of the aspiration sheath 102.

Thereafter, as shown in FIG. 9, the elongate shaft 142 of the expansion sleeve 140 may be advanced distally through the lumen of the elongate shaft 105 to radially expand or otherwise widen the distal end region 115 of the elongate shaft 105, as described above. The distal end region 115 may achieve its radially expanded configuration as the elongate shaft of the expansion sleeve 140 passes through the lumen 125 of the distal end region 115 of the elongate shaft 105. The elongate shaft 142 of the expansion sleeve 140 may have an outer diameter greater than the inner diameter of the lumen 125 of the distal end region 115 in the radially collapsed configuration, thereby exerting a radially outward force on the distal end region 115 to radially expand the distal end region 115. In other words, as the elongate shaft 142 of the expansion sleeve 140 passes into and through the distal end region 115, the portions of the elongate shaft 142 of the expansion sleeve 140 that contact the interior wall of the distal end region 115, act to expand the tubular wall of the distal end region 115 in contact with the elongate shaft 142 of the expansion sleeve 140, to radially outwardly expand the distal end region 115 into the radially expanded configuration. The syringe 185, or another syringe or other vacuum device, which may be connected to the hub 144 of the expansion sleeve 140, may thereafter be used to aspiration emboli and/or thrombus through the lumen of the expansion sleeve 140 and thus the aspiration sheath 102.

In at least some examples, portions or all of the various components of the aspiration sheath system 100 may also be doped with, made of, or otherwise include a radiopaque material including those listed herein or other suitable radiopaque materials. In some embodiments, a degree of MRI compatibility is imparted into the aspiration sheath system 100. For example, to enhance compatibility with Magnetic Resonance Imaging (MRI) machines, it may be desirable to make the various components of the aspiration sheath system 100 in a manner that would impart a degree of MRI compatibility. For example, the various components of the aspiration sheath system 100 may be made of a material that does not substantially distort the image and create substantial artifacts (artifacts are gaps in the image). Certain ferromagnetic materials, for example, may not be suitable because they may create artifacts in an MRI image. The various components of the aspiration sheath system 100 may also be made from a material that the MRI machine can image. Some materials that exhibit these characteristics include, for example, tungsten, cobalt-chromium-molybdenum alloys, nickel-cobalt-chromium-molybdenum alloys, nitinol, and the like, and others.

In some embodiments, the exterior surface of the aspiration sheath system 100 may include a coating, for example a lubricious, a hydrophilic, a protective, or other type of coating. Hydrophobic coatings such as fluoropolymers provide a dry lubricity which improves device handling and exchanges. Lubricious coatings improve steerability and improve ease of advancement within the vasculature. Suitable lubricious polymers may include silicone and the like, polymers such as high-density polyethylene (HDPE), polytetrafluoroethylene (PTFE), polyarylene oxides, polyvinylpyrrolidones, polyvinyl alcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

Some examples of suitable polymers and/or elastomers and/or elastomeric material that may be used with any of the examples disclosed herein may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX®low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be noted and can be appreciated that some of the figures are schematic in nature and are not drawn to scale. Certain features are shown larger than their scale and certain features are omitted from some views for ease of illustration.

It should also be noted that, as used in this specification and the appended claims, the singular forms include the plural unless the context clearly dictates otherwise. It should also be noted that the term “or” is generally employed in its sense including “and/or” unless the content clearly dictates otherwise.

The present disclosure has been described with reference to various specific and exemplary embodiments. Those skilled in the art will understand that changes may be made in details, particularly in matters of shape, size, material and arrangement of parts. Accordingly, various modifications and changes may be made to the examples and the embodiments. Additional or fewer components may be used, depending on the condition that is being treated by the electrosurgical ablation device and other related devices and components disclosed herein. It should be understood that many variations and modifications may be made while remaining within the spirit and scope of the present disclosure. The specifications and drawings are, therefore, to be regarded in an illustrative rather than a restrictive sense.

What is claimed is:

1. An aspiration sheath system comprising:
an expandable sheath having a proximal end region, a distal end region and a lumen extending therebetween, wherein the distal end region has a first diameter in a delivery configuration smaller than a first diameter of the proximal end region in the delivery configuration;
a dilator insertable within the lumen of the expandable sheath and through the proximal end region and the distal end region, without expanding the proximal end region and the distal end region; and
an expansion sleeve insertable within the lumen of the expandable sheath upon removal of the dilator therefrom, the expansion sleeve configured to expand the distal end region from the first diameter of the distal end region to a second diameter of the distal end region larger than the first diameter of the distal end region.

2. The aspiration sheath system of claim 1, wherein the first diameter of the distal end region is 18F or less and the second diameter of the distal end region is 20F or more.

3. The aspiration sheath system of claim 1, wherein the distal end region is retractable toward the first diameter of the distal end region after withdrawing the expansion sleeve.

4. The aspiration sheath system of claim 1, wherein the expansion sleeve maintains expansion of the distal end region in the second diameter during aspiration through the expandable sheath.

5. The aspiration sheath system of claim 1, wherein the expansion sleeve includes an aspiration lumen extending therethrough.

6. The aspiration sheath system of claim 1, wherein the distal end region includes:
an inner layer defining the lumen of the expandable sheath;
an intermediate layer surrounding the inner layer; and
an outer layer surrounding the intermediate layer.

7. The aspiration sheath system of claim 6, wherein the intermediate layer comprises a cut pattern.

8. The aspiration sheath system of claim 7, wherein the cut pattern comprises a plurality of discontinuous slits alternating with frangible links.

9. The aspiration sheath system of claim 8, wherein the frangible links are configured to break upon initial expansion of the distal end region to the second diameter.

10. The aspiration sheath system of claim 7, wherein the cut pattern extends in a helical direction along the distal end region.

11. The aspiration sheath system of claim 6, wherein the outer layer is formed of an elastomeric material configured to apply a radially inward force on the intermediate layer when the expansion sleeve is removed to return the distal end region toward the delivery configuration.

12. The aspiration sheath system of claim 1, wherein in the delivery configuration, the lumen of the expandable sheath has a first diameter throughout the proximal end region and a second diameter throughout the distal end region, the second diameter of the lumen being less than the first diameter of the lumen in the delivery configuration.

13. The aspiration sheath system of claim 12, wherein the dilator has a first diameter along a proximal end region of the dilator and the dilator has a second diameter along a distal end region of the dilator, the second diameter of the dilator being less than the first diameter of the dilator.

14. The aspiration sheath system of claim 13, wherein the first diameter of the dilator is greater than the second diameter of the lumen of the expandable sheath in the delivery configuration.

15. An aspiration sheath system comprising:
an expandable sheath having a proximal end region, a distal end region and a lumen extending therebetween, the distal end region being radially expandable from a first diameter in a delivery configuration to a second diameter in an expanded configuration;
a dilator insertable within the lumen of the expandable sheath in the delivery configuration with a distal tip of the dilator extending distally beyond the expandable sheath; and
an expansion sleeve insertable within the lumen of the expandable sheath upon removal of the dilator therefrom, the expansion sleeve configured to expand the distal end region of the expandable sheath from the first diameter to the second diameter.

16. The aspiration sheath system of claim 15, wherein the distal end region includes:
an inner layer defining the lumen of the expandable sheath;
an intermediate layer surrounding the inner layer; and
an outer layer surrounding the intermediate layer.

17. The aspiration sheath system of claim 16, wherein the intermediate layer comprises a cut pattern.

18. The aspiration sheath system of claim 17, wherein the cut pattern comprises a plurality of discontinuous slits alternating with frangible links.

19. The aspiration sheath system of claim 18, wherein the frangible links are configured to break upon initial expansion of the distal end region to the second diameter.

20. A method of using a medical device system, the method comprising:
advancing an expandable sheath through a blood vessel in a delivery configuration with a dilator disposed in a lumen of the expandable sheath and extending distally therefrom, the expandable sheath including a proximal end region and a distal end region, wherein in the delivery configuration the distal end region has a first outer diameter less than an outer diameter of the proximal end region;
removing the dilator from the lumen of the expandable sheath; thereafter, advancing an expansion sleeve through the lumen of the expandable sheath to expand the distal end region from the first outer diameter to a second outer diameter greater than the first outer diameter.
